(19) United States
(12) Patent Application Publication
(10) Pub. No.: US 2012/0064166 A1
(43) Pub. Date: Mar. 15, 2012

(54) FORMULATION COMPRISING DROSPIRENONE FOR SUBCUTANEOUS OR INTRAMUSCULAR ADMINISTRATION

(75) Inventors: Stefanie Nippe, Berlin (DE); Sascha General, Berlin (DE)

(73) Assignee: Bayer Pharma Aktienesellschaft, Berlin (DE)

(21) Appl. No.: 13/201,856

(22) PCT Filed: Feb. 12, 2010
(86) PCT No.: PCT/EP2010/051758

§ 371 Date: Nov. 30, 2011

(30) Foreign Application Priority Data
Feb. 18, 2009 (EP) 09002257.5

(51) Int. Cl. A61K 31/585; A61K 9/14; A61P 5/24; A61P 15/18

(52) U.S. Cl. 424/490; 514/173; 514/170

(57) ABSTRACT

The present invention relates to a composition comprising Drospirenone dispersed in a liquid or semi-solid lipophilic vehicle. The present invention further relates to the use of such compositions as contraceptives and for treatment of diseases, disorders and symptoms associated with deficient endogenous levels of estrogen in women.

### TECHNICAL FIELD OF THE INVENTION

The present invention relates to the field of formulations of Drospirenone (DRSP), which belongs to the group of steroids known as progestins. The formulations of the present invention are in particular useful as contraceptives and for treatment of diseases, disorders and symptoms associated with deficient endogenous levels of estrogen in women.

### BACKGROUND OF THE INVENTION

Drospirenone (DRSP) is a progestin which is known to be used in contraceptives and for treatment of diseases, disorders and symptoms associated with deficient endogenous levels of estrogen in women.

Today different formulations for administration of steroid hormones in general are known which each offer different advantages and disadvantages.

Besides the formulations efficiency the consumers experience with the ease of using said formulations is also relevant especially as correct use is necessary for the product to work satisfactory.

Aspects which are relevant when designing such formulations are among other things the mode of administration and frequency of administration. A further aspect is, that the formulation should show a low initial burst effect, as a uniform release of the drug from the formulation is desired.

Drospirenone has certain advantages compared to other progestins such as a positive effect on skin appearance and an ability to lessen complications relating to premenstrual syndrome.

However, Drospirenone has challenging physicochemical properties. It is sparingly soluble in lipophilic oil based systems. This poses a challenge when Drospirenone is designed for injection due to the limitation of administration volume.

Furthermore Drospirenone isomerizes in aqueous medium especially at acidic pH resulting in an inactivation of the compound.

There is an ongoing need for developing steroid hormone formulations so as to suit the individual consumer's needs.

One advantage of the present invention is that it results in a depot effect enabling a less than daily administration frequency.

Another aspect of the present invention to provide a formulation with an reduced burst-effect.

U.S. Pat. No. 4,016,293 relates to a sustained release depot form of the free base or pamoate salt of 2-chloro-11-(1-piperazinyl)-dibenz[b,f][1,4]-oxazepine or 2-chloro-11-(4-methyl-1-piperazinyl)-dibenz[b,f]-[1,4]oxazepine in an injectable oil for parenteral administration.

WO 93/00070 relates to sustained release formulations for maintaining low serum levels of an androgen. WO 93/00070 describes that the active compound may be administered parentally e.g. in a solution of sesame oil or olive oil and that the active compound may be any of a long list of androgens. However, WO 93/00070 does not disclose Drospirenone nor is there any disclosure of how to make formulations of androgens in an oil suitable for subcutaneous injection.

WO 2006/008640 relates to oil suspensions for oral use of a drug of low oil solubility (page 1, lines 28-29). Pregabalin and oxazolidinone antibiotics are disclosed as suitable drugs.

US 2003/0232097 relates to an oily wax suspension of a drug, such as ibuprofen, for oral administration in a soft gelatine capsule (abstract).

WO 2004/080383 relates to a pharmaceutical composition comprising a testosterone ester, castor oil and a co-solvent formulated for intramuscular injection (field of invention).

Mesigyna® is a commercially available 1 mL oily solution which contains 5 mg Estradiol valerate and 50 mg Norethisterone enanthate and which is administered intramuscularly once a month.

The presently commercially available birth control product Depo-Provera® by Pfizer for intramuscular injection and Depo-subq Provera® by Pfizer for subcutaneous injection is an aqueous suspension of medroxyprogesterone acetate (MPA).

The inventors of the present invention have found that the stability of Drospirenone in a lipophilic vehicle is increased compared to Drospirenone in an aqueous solution.

Drospirenone has a low solubility in oil and it is therefore not possible to completely dissolve the amount of Drospirenone required to obtain a contraceptive formulation. This of course makes it more difficult to administer such a suspension subcutaneously due to e.g. restrictions on the size of the syringe.

However, the inventors of the present invention further found that the mean serum concentration of Drospirenone in a rat is higher for at least 8 days after subcutaneous injection when Drospirenone is administered in peanut oil compared to administering it in an aqueous formulation and the level of Drospirenone is high enough for it to be effective for at least 14 days. Thus the depot effect of Drospirenone is improved when administered in an oily suspension compared to administering it in aqueous suspension.

Furthermore the inventors of the present invention found that a semi-solid oily Drospirenone suspension, as it can be obtained by adding an oleogelator results in a higher Area under the Curve (AUC) and a reduced burst effect.

### SUMMARY OF THE INVENTION

In one aspect the present invention relates to a composition comprising Drospirenone dispersed in a liquid or semi-solid lipophilic vehicle.

In a further aspect the present invention relates to a method of preparing a composition according to the present invention comprising:
a) mixing Drospirenone and a lipophilic vehicle
b) dispersing Drospirenone in a mixture of lipophilic vehicle and an excipient
c) loading polymeric particles with Drospirenone

In an even further embodiment the present invention relates to a method for inhibition of ovulation in women comprising administration of a composition of the present invention by injection to the women.

In an even further embodiment the present invention relates to a method for treating diseases, disorders or symptoms associated with deficient endogenous levels of estrogen in a women comprising administration of a composition of the present invention by injection to the women

### BRIEF DESCRIPTION OF THE FIGURES

FIG. 1 shows the mean concentration of Drospirenone found in the serum (measured in ng/ml) of a rat after a single subcutaneous (s.c.) injection of different formulations of Drospirenone into a rat.

The present invention will now be described in more detail in the following.

### DETAILED DESCRIPTION OF THE INVENTION

### Composition

The present invention relates to a composition comprising Drospirenone dispersed in a liquid or semi-solid lipophilic vehicle.

The term “dispersed” means in the context of the present invention that Drospirenone is not completely dissolved in the lipophilic vehicle but is present as small particles. Thus, the composition of the present invention may be described as system comprising small particles of Drospirenone in a continuous phase which is the lipophilic vehicle.

The composition of the present invention may in particular be a suspension or a colloid.

The composition of the present invention may in a particular embodiment be homogenous, i.e. Drospirenone may in particular be homogenously dispersed in the lipophilic vehicle.

The term “suspension” is used in the present invention according to the general understanding of this term. The term suspension is generally understood as a heterogeneous fluid containing solid particles that are sufficiently large for sedimentation. Usually the particles are larger than 500 nm. The internal phase (solid) is dispersed throughout the external phase (fluid) through mechanical agitation, with the use of certain excipients or suspending agents. Unlike colloids, suspensions will eventually settle. An example of a suspension is sand in water. Suspensions are classified on the basis of the dispersed phase and the dispersion medium, where the former is essentially solid while the latter may either be a solid, a liquid or a gas.

In relation to the present invention this means that in a suspension of Drospirenone in a liquid or semi-solid lipophilic vehicle Drospirenone is regarded as the dispersed phase and the lipophilic vehicle is regarded as the dispersion medium. In the present invention the dispersion medium, i.e. the lipophilic vehicle, is liquid or semi-solid. Thus the suspension of the present invention comprises solid particles of Drospirenone dispersed in a liquid or semi-solid lipophilic vehicle. When the composition of the present invention is a suspension of Drospirenone in a lipophilic vehicle the size of the Drospirenone particles are typically in the range of 0.01-500 μm, such as in the range of 0.5-200 μm, or in the range of 0.5-100 μm, or in the range of 1-100 μm, or in the range of 1-75 μm.

The term “colloid” is used in the present invention according to the general understanding of this term. In general a colloid is a mixture where one substance is dispersed evenly throughout another. The particles of the dispersed substance are only suspended in the mixture, unlike a solution, where they are completely dissolved therein. This occurs because the particles in a colloid are larger than in a solution—small enough to be dispersed evenly and maintain a homogenous appearance, but large enough to scatter light and not dissolve. Because of this dispersal, some colloids have the appearance of solutions. A colloidal system consists of two separate phases: a dispersed phase (or internal phase) and a continuous phase (or dispersion medium). The dispersed-phase particles have a diameter of between approximately 10⁻⁹-10⁻⁶ meter, such as 5-200 nanometers. Such particles are normally invisible to an optical microscope, though their presence can be confirmed with the use of an ultramicroscope or an electron microscope. Colloids are sometimes identified and characterized by their properties. For example, if a colloid consists of a solid phase dispersed in a liquid, the solid particles will not diffuse through a membrane, whereas with a solution the dissolved ions or molecules will diffuse through a membrane.

In the context of the present invention the term “liquid” means that the lipophilic vehicle is liquid at room temperature and 1 atm pressure and/or has a melting point below 40° C. Such liquid lipophilic vehicles may be selected from list shown below.

Room temperature generally refers to a temperature between 18-25° C., such as 20° C., at 1 atm.

### Drospirenone

Drospirenone is a synthetic progestin which is an analog of spironolactone, and it has a molecular weight of 366.5 and the molecular formula C₂₄H₃₀O₃. It is also described with the chemical formula 6β,7β; 15β,16β-dimethylene-3-oxo-17α-pregn-4-ene-21,17-carbolactone.

Apart form the active substance itself, it is envisaged that an ester or prodrug of Drospirenone may be employed in the present composition, e.g. an oxyiminopregnane carbolactone as disclosed in WO 98/24801.

Drospirenone, which may be prepared substantially as described in, e.g., U.S. Pat. No. 4,129,564, or WO 98/06738, is a sparingly soluble substance in water and aqueous buffers at various pH values. Furthermore, Drospirenone is rearranged to an inactive isomer under acid conditions. It has been shown (WO 01/15701) that in order to obtain a good bioavailability of the compound, it should advantageously provided in a form that promotes rapid dissolution thereof.

In a particular embodiment the Drospirenone of the present invention may be micronized Drospirenone. When used herein, the term “micronised” is intended to mean that the particle size distribution is so that at least 90% of the particles have a particle diameter of less than 30 μm (calculated from the volume distribution curve under the presumption of spherical particles), i.e. a d₉₀ value of at the most 30 μm. Therefore, it is important to note that whenever the terms “particle size distribution”, “particle diameter”, “d₉₀”, etc. are used herein it should be understood that the specific values or ranges used in connection therewith are always meant to be determined from the volume distribution curve under the presumption of spherical particles.

### Lipophilic Vehicle

The term “lipophilic” generally refers to the ability of a compound to dissolve in fats, oils, lipids and non-polar solvents such as hexane or toluene. Typically lipophilicity is described by the partition coefficient, which is the ratio of concentrations of an un-ionized compound between two immiscible solvents at equilibrium, wherein the solvents chosen are water and octanol. The partition coefficient may then be calculated by the following formula:

log P_{oct/wat}=log((solute)_{octano}/(solute)_{un-inonized water})

The term “solute” refers in this context to the compound for which the partition coefficient is to be determined.

To measure the partition coefficient of ionisable solutes the pH of the aqueous phase is adjusted such that the predominant form of the compound is un-ionized. There are different methods of determining the partition coefficient but the classical method is the so-called shake flask method which consists of dissolving some of the solute in question in a volume of octanol and water, then measuring the concentration of the solute in each solvent. The most common method of measuring the distribution of the solute is by UV/VIS spectroscopy.

Other methods of measuring the partition coefficient include HPLC and electrochemical methods.

In the context of the present invention a liphophilic vehicle is a compound for which log P_{oct/wat}>0; at 25° C., such as log P_{oct/wat}>1; at 25° C., or log P_{oct/wat}>2; at 25° C., or log P_{oct/wat}>3; at 25° C., or log P_{oct/wat}>4; at 25° C., or log P_{oct/wat}>5; at 25° C.

The lipophilic vehicle of the present invention may be a liquid or a semi-solid. Examples of suitable lipophilic vehicles include but are not limited to lipids, fats, mono-, di-, or tri-acylglycerides like medium chain triglycerides, fatty acid esters, oils or organic solvents.

Medium chain triglycerides are medium chain; i.e. with 6 to 12 carbon atoms, fatty acid esters of glycerol. In a particular embodiment the medium chain triglyceride may be Caprylic/Capric Acid Triglyceride such as the commercially available Myritol® 318 from Cognis which is also used in the present examples. For example suitable oils may be mineral oils, animal oils, or vegetable oils.

Examples of animal oils include but are not limited to fish oils, such as cod liver of menhaden oil. Examples of vegetable oils include but are not limited to castor oil, sesame oil, castor oil, peanut oil, cottonseed oil, coconut oil, soybean oil, palm, oil, sunflower oil, thistle oil, rapeseed/canola oil, linseed oil, almond oil, maize oil, olive oil, safflower oil, corn oil, avocado oil, or any combination of two or more of these oils.

In another embodiment the lipophilic vehicle is an organic solvent. Examples of suitable organic solvents include but are not limited to silicone oil, lysolipids, phospholipids, crospovidone, cyclomehtinon, dibutyl phthalate, dibutyl sebacate, dimethicone, ethylene glycol palmitstearate, glyceryl esters, such as glyceryl monooleate, propylene carbonate, simethicone, medium chain alkanes, derivatives of alkanes such as alcohol, aldehydes, sulfonates, esters, ethers, ethyloxates, benzyl alcohol, benzyl benzoate, dimethyl acetamid, dimethyl sulfoxide, glycofurol, ethyl oleate, isopropyl myristate, isopropyl palmitate, n-methylpyrrolidone, oleyl oleate, polyethylene glycol, polyetherpolyols, propylene glycol, triacetin, α-D,L-Tocopherol, alpha-tocopheryl ester, polyethylene castor oil derivates, oleic acid, glycerol or any combination of two or more of these organic solvents.

In a preferred embodiment the lipophilic vehicle is castor oil, e.g. refined castor oil, sesame oil, peanut oil, or medium chain triglyceride (MCT), e.g. the MCT Caprylic/Capric Acid Triglyceride.

In one embodiment the composition of the present invention may comprise two or more different lipophilic vehicles. For example it is foreseen that the lipophilic vehicle in may comprise an organic solvent and an oil, such as any combination of those organic solvents and oils described above. For example the lipophilic vehicle may be a mixture of castor oil and benzyl benzoate.

The lipophilic vehicle may in some embodiments be semi-solid. Typically such semi-solids may be composed of a lipophilic liquid and an excipient which form a gel structure.

Semi-solids have properties between those of a solid and those of a liquid. Examples of semi-solid formulation are gels.

Most oleogels, also called hydrophobic gels, are prepared by heating a mixture of a gelator and a liquid lipophilic component to form a solution/dispersion, followed by cooling, which sets into a gel. A gel is a semi-solid vehicle. In a gel the sedimentation of particles can be avoided.

Examples of suitable lipophilic liquids include any of those described above.

Examples of suitable gelators include but are not limited to Methyl cholate, Cholesteryl fatty acid ester like cholesteryl stearate, polyvalent metal salts of fatty acids e.g. aluminium stearate, sorbitan fatty acid ester like sorbitan monolaurate or sorbitan monostearate, glycerol fatty acid ester, fatty acid ester of carbohydrates like dextrin palmitate (Rheopearl®), hydrophobic colloidal anhydrous silica and colloidal silicon dioxide.

Other examples of suitable gelators include but are not limited to n-alkanes, fatty acids, 1,3:2,4-di-O-benzylidene-D-sorbitol, anthryl derivates, macrocyclic gelators e.g. calixarens, ALS compounds (aromatic moiety attached to a steroidal group by linker segment), cyclo(peptides), peptide derivates, amid and urea compounds, bisurea compounds, bisamides, bolaform amides derived from amino acids, fatty acid derivates of L-alanine, Lecithin, phosphatidylcholin, amino acids, steroids, organometallic compounds, nucleotides, dentrimers, 3,5-Diaminobenzoate, cholesterol derivate, sugar derivates, crown ether phtalocynanine, glycerol fatty acid ester like Dynasan®, poly(ethylene), crosslinked poly(acrylic acid), copolymers of methacrylic acid and methmethylacrylic acid, alkylated polyglycerol methacrylate, glutamated based gelators, polysorbates.

The composition of the present invention may in a particular embodiment have a concentration of Drospirenone in the range of 20-250 mg/ml.

Drospirenone is mainly used in contraceptives and in the treatment of diseases, disorders and symptoms associated with deficient endogenous levels of estrogen in women and it is generally administered in combination with an estrogen.

Hence in a preferred embodiment the present invention further comprises an estrogen.

If the composition of the present invention is used as a contraceptive said Estrogen may in particular be ethinylestradiol.

If the composition of the present invention is used for treatment of diseases, disorders and symptoms associated with deficient endogenous levels of estrogen in women said estrogen may be natural or a synthetic derivative thereof. In preferred embodiments, the estrogen is selected from the group consisting of estradiol, estradiol sulfamates, estradiol valerate, estradiol benzoate, ethinyl estradiol, estrone, estriol, estriol succinate and conjugated estrogens, including conjugated equine estrogens such as estrone sulfate, 17β-estradiol sulfate, 17α-estradiol sulfate, equilin sulfate, 17β-dihydroequilin sulfate, 17α-dihydroequilin sulfate, equilenin sulfate, 17β-dihydroequilenin sulfate and 17α-dihydroequilenin sulfate or mixtures thereof. Particularly interesting estrogens are selected from the group consisting of estradiol, estradiol sulfamates, estradiol valerate, estradiol benzoate, estrone, and estrone sulfate or mixtures thereof, notably estradiol, estradiol valerate, estradiol benzoate and estradiol sulfamates. Most preferred is estradiol or estradiol sulfamates, particularly estradiol.

In certain embodiments of the invention, the composition may comprise more than one estrogen.

In one embodiment the composition according to the present invention may further comprise one or more excipients. Examples of suitable excipients include but are not limited to surfactants, suspending agents, gelling agents, antioxidants, dyes, analgetic excipients or a combination of two or more of these.

Excipients which are added to the lipophilic vehicle can have different functions. They can be used for example as a suspending agent or surface-active agent.

An example is sorbitan monostearate which is non-ionic surfactant and suspending agent (S. Murdan, Organogels in drug delivery, Expert Opin. Drug Deliv. 2 (2005) 489-505; Handbook of Pharmaceutical Excipients, 2003). Aerosil®200 is used as suspending agent in a concentration of 2-5% in oily vehicle to avoid sedimention and is used as gelling agent of 8 to 12% in oily vehicle (Fiedler Lexikon der Hilfsstoffe, Editio Cantor Verlag, 2002).

Examples of suitable surfactants may be found in Handbook of Pharmaceutical Excipients 2003. Such surfactants may in particular be non-ionic surfactants, amphoteric surfactants, anionic surfactants or cationic surfactants. In particular the surfactant may be lecithine, polyoxyethylene sorbitan fatty acid esters, such as polyoxyethylene(5)sorbitanmonooleate, polyoxyethylene glycol fatty acid esters, such as polyoxyl 6 stearate, polyoxylethylene castor oil derivatives, such as polyoxyl 35 castor oil, sorbitan fatty acid esters, such as sorbitan sesquioleate, sorbitan monostearate or sorbitan monolaurate, glyceryl mono- and diesters with fatty acids, such as glyceryl monostearate, tocopheryl acetate, such as D,L-α-tocopheryl acetate, cholesteryl fatty acid esters, such as cholesteryl oleate, cholesteryl nonanoate or cholesteryl stearate, cholic acid ester, such as methyl cholate, or cholic acid salt, such as sodium cholate. Other examples of suitable surfactants which may used in the composition of the present invention include but are not limited to polyvinylpyrrolidone, fatty alcohol, such as stearyl alcohol or stearoyl alcohol, poloxamer, such as poloxamer 124, and polyoxyethylene alkyl ethers, such as Brij 30, Brij 93 or Brij 72. Suspending agents helps to avoid or reduce sedimentation of the Drospirenone in the composition, which is in particular relevant for long-term storage of the composition. As described above suspending agents may also form a gel in a lipophilic liquid so that the lipophilic vehicle is a semi-solid. Examples of suitable suspending agents which may be used in the composition of the present invention include but are not limited to hydrophobic colloidal anhydrous silica and colloidal silicon dioxide, methyl cholate, cholesteryl fatty acid ester like cholesteryl stearate, cholesteryl oleate and cholesteryl nonanoate, polyvalent metal salts of fatty acids e.g. aluminium stearate, sorbitan fatty acid ester like sorbitan monolaurate and sorbitan monostearate and glycerol fatty acid ester like glycerol monostearate. Examples of other suitable suspending agents which may used in the composition of the present invention include but are not limited to n-alkanes, fatty acids, 1,3:2,4-di-O-benzylidene-D-sorbitol, anthryl derivates, macrocyclic gelators e.g. calixarens, ALS compounds (aromatic moiety attached to a steroidal group by linker segment), cyclo(peptides), peptide derivates, amid and urea compounds, bisurea compounds, bisamides, bolaform amides derived from amino acids, fatty acid derivates of L-alanine, Lecithin, phosphatidylcholin, amino acids, steroids, organometallic compounds, nucleotides, dentrimers, 3,5-Diaminobenzoate, cholesterol derivate, sugar derivates, crown ether phtalocynanine, Dynasan®, poly(ethylene), crosslinked poly(acrylic acid), copolymers of methacrylic acid and methmethylacrylic acid, alkylated polyglycerol methacrylate, glutamated based gelators, fatty acid ester of carbohydrates like dextrin palmitate (Rheopearl®), polysorbates, polyester, such as polylactide, polyglycolide, poly-lactide-glycolide (also know as poly(lactide-co-glycolide respectively_PLGA), polycaprolactone, carboxymethylcellulose, ethylcellulose and gelatine, PEG copolymer, albumine. An example of a suitable antioxidant is D,L-α-tocopherol acetate. Other examples of suitable antioxidants which may be used in the composition of the present invention include but are not limited to α-tocopherol, butylated hydroxyanisol and butylated hydroxytoluene.

In a preferred embodiment the composition of the present invention comprises one or more of the following excipients: methyl cholate, glycerol monostearate, cholesteryl oleate, cholesteryl nonanoate, cholesteryl stearate, lecithin, (such as L-α-Lecithin, e.g. from soybean), α-tocopherol acetate (such as D,L-α-tocopherol acetate), sorbitan monostearate, sorbitan sesquioleate, sorbitan monolaurate, polyoxyethylene(5)sorbitan monooleate, polyoxyethylene(6)stearate, castor oil, polyoxyethylene ether, hydrophobic colloidal anhydrous silica (such as Aerosil®972), colloidal silicon dioxide (such as Aerosil®200), or polyvalent metal salts of fatty acids e.g. aluminium stearate.

If one or more of these excipients are present the lipophilic vehicle may in particular be medium chain triglycerides, such as the medium chain triglyceride Caprylic/Capric Acid Triglyceride.

An example of a suitable analgetic agent is benzyl alcohol.

In one embodiment the composition according to the present invention the DRSP respectively Ethinylestradiol microparticles are encapsulated in a biodegradable polymer before suspended in a lipophilic vehicle.

Biodegradable polymers which may be used to encapsulate DRSP or Ethinylestradiol include but are not limited to poly(lactide-co-glycolide), poly(lactide), and poly(alkylcyanoacrylate) such as poly(butylcyanoacrylate) (PBCA).

### Manufacture of the Composition

The present invention also relates to a method of preparing a composition of the present invention, said method comprising the steps of:
a) mixing Drospirenone and a lipophilic vehicle
b) dispersing Drospirenone in a mixture of lipophilic vehicle and an excipient

If the lipophilic vehicle is liquid step a) may for example be performed by adding 10 mg-10 g of Drospirenone to 0.5 ml-5 L lipophilic vehicle.

However, step a) may also be performed by adding the lipophilic vehicle to Drospirenone.

If the lipophilic vehicle is semi-solid and e.g. composed of a lipophilic liquid and an excipient these components may typically be mixed prior to adding Drospirenone to the lipophilic vehicle.

Methods of doing the actual mixing of Drospirenone and the lipophilic vehicle are known to a person skilled in the art and may be performed as described in the examples. For example such methods include but are not limited to mixing or blending with a roller mixer, blending, e.g. with a magnetic stirrer, pestle or twisted paddle stirrer, sonication, or vortex mixer.

The method of the present invention may in some embodiments comprise a further step b):

b) separation of the composition obtained in step a).

In some cases it is necessary to heat up the mixture of lipophilic vehicle and excipient. After cooling Drospirenone is added.

As described above the composition of the present invention may in some embodiments further comprise other components such as a suspending agent, excipient or any of the other components mentioned above. When to add such components in the composition depends on the exact component and it is within the knowledge of a person skilled in the art to mix such components. For example if a lipophilic suspending agent is to be included in the composition this may typically be mixed with the lipophilic vehicle prior to adding Drospirenone to the lipophilic vehicle.

### Use of the Composition

The present invention also relates to use of a composition according to the present invention as a contraceptive and for the treatment of diseases, disorders and symptoms associated with deficient endogenous levels of estrogen in women.

Hence the present invention also relates to a method for inhibition of ovulation in a women comprising administration by injection of a composition according to the present invention.

The present invention also relates to a method for treating diseases, disorders, or symptoms associated with deficient endogenous levels of estrogen in a women comprising administration by injection of a composition according to the present invention.

Thus the present invention also relates to a composition according to the present invention for the use as a medicament.

In a preferred embodiment the composition is for inhibition of ovulation in a women of for treating diseases, disorders, or symptoms associated with deficient endogenous levels of estrogen in a women

In the present context, the term cycle itself or when associated with the term menstrual is intended to mean the number of days between menses in a woman. It can range from 21-31 days, typically 28 days.

In the present context, the term menopause is understood as the last natural (ovary-induced) menstruation. It is a single event and a result of an age-dependent dysfunction of the ovarian follicles. Menopause results from the ovaries decreasing their production of the sex hormones estrogen and progesterone. When the number of follicles falls below a certain threshold (a bleeding threshold), the ovaries can no longer produce mature follicles and sex hormones. The ability to reproduce capability ends with menopause.

The peri-menopausal phase begins with the onset of climacteric symptoms when the cycle becomes irregular and ends one year after menopause. The end of peri-menopausal phase can be identified after a protracted period of time without bleeding. Post-menopause is the phase that begins at menopause and continues until death.

One principal aim of hormone replacement therapy is to restore levels of the sex steroid hormones in naturally or prematurely pre-menopausal, menopausal and post-menopausal women or to establish these levels in hypogonadal females.

Deficient levels of estrogen can occur for a variety of reasons. The composition can be such that it is adequate for deficient levels of estrogen, regardless of the cause. Causes anticipated by the therapy are, but not limited to, natural menopause, peri-menopause, post-menopause, hypogonadism, castration or primary ovarian failure.

Low levels of estrogen, irrespective of the cause, lead to an overall decreased quality of life for women. Symptoms, diseases and disorders range from merely being inconvenient to life threatening. The composition of this therapy anticipates the effective alleviation of all physiological and psychological signs of estrogen deficiency.

Transient symptoms, such as vasomotor signs and psychological symptoms are certainly embodied with the realm of therapy. Vasomotor signs comprise but are not limited to hot flushes, sweating attacks such as night sweats, and palpitations. Psychological symptoms of estrogen deficiency comprise, but are not limited to, insomnia and other sleep disorders, poor memory, loss of confidence, mood changes, anxiety, loss of libido, difficulties in concentration, difficulty in making decisions, diminished energy and drive, irritability, and crying spells.

The treatment of the aforementioned symptoms can be associated with the peri-menopausal phase of a woman's life or after, sometimes long after menopause. It is anticipated that the invention is applicable to these and other transient symptoms during the peri-menopausal phase, menopause, or post-menopausal phase. Moreover, the aforementioned symptoms can be alleviated if the cause of the estrogen deficiency is hypogonadism, castration or primary ovarian failure.

In another embodiment of the invention, the therapy is used for the treatment of permanent effects of estrogen deficiency. Permanent effects comprise physical changes such as urogenital atrophy, atrophy of the breasts, cardiovascular disease, changes in hair distribution, thickness of hair, changes in skin condition and osteoporosis.

Urogenital atrophy, conditions associated with it such as vaginal dryness, increase in vaginal pH and subsequent changes in flora, or events which lead to such atrophy, such as decreases in vascularity, fragmentation of elastic fibres, fusion of collagen fibres, or decreases in cell volume are symptoms thought to be particularly relevant to this therapy. Furthermore, the invention is thought to be relevant to other urogenital changes associated estrogen deficiency such as decreases in the length and/or diameter of the vagina, decreases mucus production, changes in cell population, decreases in glycogen production, decreases in growth of lactobacilli or increases in growth of streptococci, staphylococci, or coliform bacilli. Other associated changes that are thought to be preventable by the invention are those that may render the vagina susceptible to injury or infection, such as exudative discharges, vaginitis, and dyspareunia. Furthermore, infections of the urinary tract and incontinence are other common symptoms associated with lowered estrogen levels.

Other embodiments of the invention include the prevention or alleviation of physical changes associated with estrogen deficiency, such as changes in the skin, changes in hair distribution, thickness of hair, atrophy of the breasts, or osteoporosis.

The prevention and management of osteoporosis, most notably post-menopausal osteoporosis, is a particularly interesting embodiment of the invention.

Furthermore, bone demineralisation, reduction of bone mass and density, thinning and interruption of trabeculae, and/or consequent increase in bone fractures or bone deformations are thought to be particularly relevant. The prophylactic treatment of osteoporosis is an interesting therapeutic application of the invention.

A particularly interesting embodiment of the invention comprises the use of the composition for lessening the frequency, persistence, duration and/or severity of hot flushes, sweating attacks, palpitations, sleep disorders, mood changes, nervousness, anxiety, poor memory, loss of confidence, loss of libido, poor concentration, diminished energy, diminished drive, irritability, urogenital atrophy, atrophy of the breasts, cardiovascular disease, changes in hair distribution, thickness of hair, changes in skin condition and osteoporosis, most notably hot flushes, sweating attacks, palpitations, sleep disorders, mood changes, nervousness, anxiety, urogenital atrophy, atrophy of the breasts or for the prevention or management of osteoporosis.

Independent of its use the composition according to the present invention it is foreseen that the composition is to be administered by injection. The term “administration by injection” is meant to encompass any form for injection into a muscle or subcutaneous injection. The preferred form of injection is by subcutaneous injection.

The volume that can be injected intramuscularly is known to affect the release rate of an active principle from a vehicle. An injection volume of 1 mL is generally considered as the maximum volume that can be administered by on single subcutaneous injection to one injection. Similarly, the maximum injection intramuscular injection volume is generally considered to be 5 mL

When the injection of volumes greater than those maximum values is required, the injection volume needs to be divided into two or more separate injections to different injection sites. However, multiple injections for the administering of one dose are generally not preferred because of the inconvenience conferred to the female.

Furthermore, the injection of a single dose to one injection site offers great advantages in controlling the release rate of an active principle, rather than multiple injections of divided single doses. Thus in interesting embodiments the subcutaneous injection volume is typically in the range of 0.2-1 mL, while the intramuscular injection volume is typically in the range of 1-5 mL.

The composition may be suitable formulated as a unit dose form such as a unit dose intended for being injected as one single dose.

Independent of whether composition of the present invention is administered subcutaneously or intramuscularly the injected single dose of Drospirenone may typically be in the range of 1-250 mg, such as between 30-200 mg of Drospirenone.

The compositions of the present invention are expected to administer less frequently than oral contraceptives which are generally administered on a daily basis.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1

### Long-Term Stability Test

The following formulations (Samples 1.1-1.5) have been prepared to investigate the long-term stability (chemical stability and particle size distribution).

### Starting Materials:

Drospirenone, micronized (DRSP mikro 15, Syn. C, Var. 3, ZK 30595, Stoffnummer 3123163, charge 85302620)
Medium chain triglyceride (Myritol® 318 PH, Cognis GmbH, Düsseldorf, Germany)
Sesame oil (from Sesamum indicum, Fluka Chemie AG, Buchs, Switzerland)
Castor oil, refined, puriss. (Riedel-de-Haen, Sigma Aldrich Laborchemikalien GMBH, Seelze, Germany)
Peanut oil (Charge 13786159)

Sample 1.1: Drospirenone in water 500 mg micronized Drospirenone was suspended in 5 ml MilliQ water saturated with Drospirenone. The suspension was then mixed for two minutes using a vortex mixer (Heidolph, REAK 2000, level 4) and was blended with a roller mixer (Britze, DA II) for 24 h with 870 U/min.

The suspension was stored at room temperature prior to use.

Sample 1.2: Drospirenone in castor oil 2000 mg micronized Drospirenone was added to 20 ml refined castor oil saturated with Drospirenone. The suspension was mixed for 2 min using vortex mixer (Heidolph, REAK 2000, level 4) and was blended with a roller mixer (Britze, DA II) for 24 h with 870 U/min.

The suspension was stored tightly closed in wide-neck brown glass flask in an environmental chamber at 25° C. and a relative humidity of 60%.

Sample 1.3: Drospirenone in sesame oil 2000 mg micronized Drospirenone was added to 20 ml sesame oil saturated with Drospirenone. The suspension was mixed for 2 min using vortex mixer (Heidolph, REAK 2000, level 4) and was blended with a roller mixer (Britze, DA II) for 24 h with 870 U/min.

The suspension was stored tightly closed in wide-neck brown glass flask in an environmental chamber at 25° C. and a relative humidity of 60%.

Sample 1.4: Drospirenone in medium chain triglycerides 2000 mg micronized Drospirenone was added to 20 mL of the commercially available medium chain triglyceride Caprylic/Capric Acid Triglyceride known as Myritol®318 PH which was saturated with Drospirenone. The suspension was mixed for 2 min using vortex mixer (Heidolph, REAK 2000, level 4) and was blended with a roller mixer (Britze, DA II) for 24 h with 870 U/min.

The suspension was stored tightly closed in wide-neck brown glass flask in an environmental chamber at 25° C. and a relative humidity of 60%.

Sample 1.5: Drospirenone in peanut oil

5.4 mg Drospirenone (median particle size 50 μm) was added to 45.0 ml peanut oil. The suspension was blended with a magnetic stirrer (Ika-Werke, RCT, Germany).

### Example 1a

### Chemical Stability of Drospirenone in Water and Oil

Drospirenone and Isodrospirenone were detected by HPLC/UV. Prior to running the samples on the HPLC each of the suspensions was prepared according to the following procedure:

### Aqueous Suspension of Drospirenone

The suspension (n=3) was mixed for 30 s using vortex mixer (Heidolph, REAK 2000; level 4). 2 times 1 ml was pipetted into an Eppendorf tube with filter cartridge. The sample was then centrifuged for 10 min at 7500 U/min (centrifuge: Sigma Laborzentrifugen ZK15). 1 ml of the separated Drospirenone solution was mixed with 1 ml Acetonitril. The amount of Drospirenone and Isodrospirenone was analyzed by HPLC/UV.

### Oil-Based Suspension of Drospirenone

The suspension (n=3) was mixed 30 sec using vortex mixer (Heidolph, REAK 2000; level 4). 0.5 ml was pipetted into an Eppendorf tube. The sample was centrifuged twice for 10 min at 7500 U/min (centrifuge: Sigma Laborzentrifugen ZK15). 3×50 μl of the supernatant was pipetted into a volumetric flask and each mixed with 10 ml acetonitrile. The amounts of Drospirenone and Isodrospirenone were analyzed by HPLC/UV.

### HPLC/UV Test Conditions:

HPLC: Agilent, Hewlett Packard series 1100
Column: ODS Hypersil (length 6 cm, inner diameter 4.6 mm)
Mobile phase: 60/40 water/acetonitril
Column temperature: approximately 22° C.
Injection volume: 100 μl
Run time: 10 min
Detector: UV detector
Wave length: 270 nm

The degradation of Drospirenone into Isodrospirenone was measured relatively as the ratio of the area of the peak (or peak area) corresponding to Drospirenone and the area of the peak corresponding to isodrospirenone.

After 14 days storage Isodrospirenone could be detected in the aqueous suspension of Drospirenone (sample 1.1.) with a mean ratio of Drospirenone to Isodrospirenone being 8.89.

No Isodrospirenone could be detected after 4 months storage of Drospirenone in castor oil (sample 1.2.), sesame oil (sample 1.3.) or medium chain triglycerides (sample 1.4.).

Similarly no Isodrospirenone could be detected after 2 months storage of Drospirenone in peanut oil (sample 1.5.).

These data show that Drospirenone is chemically stable for a longer period of time suspended in oily medium than in water.

### Example 1b

### Particle Size Distribution During Storage of DRSP in Oil

The particle size distribution of Drospirenone microcrystals was tested using an image analysis counting and sizing system.

The particle sizes were measured 1 day after preparation and before storage in the environmental chamber as well as on day 7, 13, 27, 77 and 178.

Before the analysis the suspension was mixed with a roll mixer at 870 U/min for 12 hours.

The analysis was carried out with the following equipment:
Microscope: Olympus BX50
Camera: Olympus U-CMAD-2 (Adapter)
Analysis software: analysis 5.0, Soft Imaging System GmbH
Adjustment: Magnification 500×, Extended Focal Imaging, Red Filter, Measuring “Ausdehnung Auβen Max”, Minimum 6 Pixel.

The distribution of the particle size of Drospirenone microcrystals was measured in Drospirenone suspended in castor oil, sesame oil and medium chain triglycerides (samples 1.2., 1.3. and 1.4.).

The results in table 1 show that the median particle sizes of Drospirenone in each oily suspensions varies between 2-4 μm and is constant over a time period of 178 days.

TABLE 1

| | | | Sample 1.4. |
|---|---|---|---|
| | Sample 1.2. | Sample 1.3. | (Medium chain |
| | (castor oil) | (sesame oil) | triglycerides) |
| Time (days) | Median particle size (μm); n = 3 | | |
| 1 | 3-4 μm | 3-4 μm | 3-4 μm |
| 7 | 3-4 μm | 3-4 μm | 3-4 μm |
| 13 | 3-4 μm | 3-4 μm | 3-4 μm |
| 27 | 4-5 μm | 2-3 μm | 3-4 μm |
| 77 | | 3-4 μm | 4-5 μm |
| 178 | 3-4 μm | 3-4 μm | 3-4 μm |

### Example 2

### Analysis of the Sedimentation of DRSP During Storage

The following formulations (Sample 2.1-2.2) have been prepared to investigate the sedimentation of Drospirenone microcrystals during storage.

### Sample 2.1: Oil-Based Drospirenone Microcrystal Suspension

Drospirenone, micronized (Drospirenone mikro 15, Syn. C, Var. 3, ZK 30595, Stoffnummer 3123163, charge 85302620)
medium chain triglyceride (Myritol 318 PH)

1.4 g Drospirenone was added to 10 ml oil. The drug substance was suspended in the vehicle for 1 min at 1×10% cycle, 100% power using ultrasound device using (Bandelin, Sonopuls, HD2070).

The formulation was stored at room temperature in a test tube.

### Sample 2.2: Drospirenone Oleogel

Drospirenone, micronized (Drospirenone mikro 15, Syn. C, Var. 3, ZK 30595, Stoffnummer 3123163, charge 85302620)
medium chain triglyceride (Myritol 318 PH)

### Excipients (E):

E.1.: Cholesteryl oleate, 97% (Alfa Aesar, lot 17854)
E.2.: Methyl cholate, 98+% (Alfa Aesar, lot 10049681)
E.3.: Silica, hydrophobic colloidal anhydrous (Aerosil®972 Pharma, Degussa, control number 3156111323)
E.4.: Colloidal silicon dioxide (Aerosil®200 Pharma, Degussa, Sample, control number 3157042814)
E.5. or E.6.: Dextrin palmitate derivatives (Rheopearl® TL2 or Rheopearl® KL2),
E.7.: Dextrin (palmitate/ethyl hexanoate) (Rheopearl® TT2) (f S. Black GmbH, Duisburg, Germany)

One of the a.m. excipients (E.1-E.7) and medium chain triglycerides were mixed for 1 min using a vortex mixer (Heidolph, REAK 2000; level 4) and was sonicated at 5×10% cycle, 100% power using ultrasound device (Bandelin, Sonopuls, HD2070) until getting a clear solution which after cooling results into a gel. After cooling for 30 min 10 ml gel was added to 1.4 g Drospirenone. The drug substance was suspended in the vehicle for 1 min at 1×10% cycle, 100% power using ultrasound device (Bandelin, Sonopuls, HD2070).

The formulation was stored at room temperature in a test tube.
E.8.: aluminium stearate (Fluka, Sigma-Aldrich Chemi GmbH, Steinheim, lot 1333259)

Aluminium stearate (E.8) and medium chain triglycerides were mixed for one minute using a vortex mixer (Heidolph, REAK 2000; level 4). The mixture is heated up to 200° C. on a heating plate. After cooling for 2 h 10 ml gel was added to 1.4 g Drospirenone. The drug substance was suspended in the vehicle for 1 min at 1×10% cycle, 100% power using ultrasound device using (Bandelin, Sonopuls, HD2070).

The formulation was stored at room temperature in a test tube.

### Sedimentation of Drospirenone Microcrystals During Storage

The sedimentation velocity of Drospirenone microcrystals during storage was examined as well as the effect the presence of different suspending agents have on the sedimentation velocity.

For analyzing sedimentation velocity the suspension was photographed after preparation as well as 2, 6, 12, 27 and 80 days after preparation. The level of sediment and supernatant was measured at each time point.

The software used for measuring the level of sediment and supernatant was Axio Vision 4.5, Carl Zeiss Imaging Solutions.

Sedimentation velocity of each of the a.m. formulations (Sample 2.1-2.2) was measured. The results are shown in Table 2 were the percentage of supernatant over time is shown for each of the formulations.

TABLE 2

| | | | Supernatant |
|---|---|---|---|
| | Concentration of | Concentration of | after day 60 |
| Excipient | Excipient | Drospirenone | [%] |
| Methyl cholate | 15 mg/ml | 70 mg/ml | <5% |
| Cholesteryl stearate | 100 mg/ml | 70 mg/ml | <5% |
| Cholesteryl oleate | 400 mg/ml | 70 mg/ml | <5% |
| Aerosil ® 972 | 100 mg/ml | 70 mg/ml | <5% |
| Aerosil ® 200 | 30 mg/ml | 70 mg/ml | <5% |
| Aluminium stearate | 30 mg/ml | 70 mg/ml | <5% |
| Rheopearl ® TT | 50 mg/ml | 70 mg/ml | <5% |
| Rheopearl ® TL | 50 mg/ml | 70 mg/ml | <5% |
| Rheopearl ® KL | 50 mg/ml | 70 mg/ml | <5% |

Sample 2.3: 240 mg drospirenone, micronized (DRSP mikro 15, Syn. C, Var. 3, ZK 30595, Stoffnummer 3123163, charge 85302620)
4 ml medium chain triglyceride (Myritol 318 PH)

4 ml medium chain triglyceride was added to 240 mg drospirenone. The compounds were been mixed for one minute using a vortex mixer (Heidolph, REAK 2000; level 4). Afterwards the suspension was sonicated for 30 sec at 5×10% cycle, 100% power using ultrasound device (Bandelin, Sonopuls, HD2070).

The formulation was stored at room temperature in a test tube.

### Sample 2.4:

240 mg drospirenone, micronized (DRSP mikro 15, Syn. C, Var. 3, ZK 30595, Stoffnummer 3123163, charge 85302620)
200 mg excipient
4 ml medium chain triglyceride (Myritol 318 PH)

### Excipients:

E.2. Cholesteryl oleate, 97% (Alfa Aesar, lot 17854)
E.3. Cholesteryl nonanoate, 97% (ABCR, lot 1001806)
E.5. L-α-Lecithin (from soybean) (Calbiochem, lot B69528)
E.8. Sorbitan sesquioleate (Sigma Aldrich, lot 044K0024)
E.9. Polyoxyethylen(5)sorbitan monooleate (Tween® 81, Sigma Aldrich, lot 082H0300)
E.10. Polyoxyethylen(6)stearate

4 ml medium chain triglyceride was added to 200 mg excipient and 240 mg drospirenone. The compounds were been mixed for one minute using a vortex mixer (Heidolph, REAK 2000; level 4). Afterwards the suspension was been sonicated for 30 sec at 5×10% cycle, 100% power using ultrasound device (Bandelin, Sonopuls, HD2070).

The formulation was stored at room temperature in a test tube.

TABLE 2

| | day 2 | day 6 | day 12 | day 27 | day 80 |
|---|---|---|---|---|---|
| Excipient | Supernatant [%] | | | | |
| Tween 81 | 0.00 | 0.00 | 23.04 | 22.33 | 39.95 |
| Polyoxyethylen 6 stearate | 0.00 | 5.15 | 23.63 | 26.41 | 38.93 |
| Cholesteryl nonanoate | 8.06 | 14.60 | 32.09 | 44.15 | 46.68 |
| Lecithin | 0.00 | 7.92 | 30.27 | 43.56 | 48.06 |
| Cholesteryl oleate | 16.70 | 18.61 | 30.15 | 45.05 | 50.89 |
| Span 83 | 11.28 | 17.76 | 33.24 | 51.15 | 52.09 |
| without stabilizer | 54.01 | 52.90 | 53.03 | 53.03 | 53.03 |

### Example 3

### Investigation the In Vivo Release Kinetics

The following formulations (sample 3.1-3.4) have been prepared to investigate the in vivo release kinetic of Drospirenone.

### Sample 3.1: Aqueous Drospirenone Microcrystal Suspension

Drospirenone (micronized, 20, 50 or 110 μm respectively) was suspended using a magnetic stirrer (Ika-Werke, RCT, Germany) in isotonic NaCl solution containing 0.25% Tween 80 and 1% Klucel LF.

### Sample 3.2: Oil-Based Drospirenone Microcrystal Suspension

5.4 mg Drospirenone (median particle size 50 μm) was added to 45.0 ml peanut oil. The suspension was been blended with a magnetic stirrer (Ika-Werke, RCT, Germany)

### Sample 3.3: Drospirenone Oleogel

Methyl cholate was dispersed in medium chain triglycerides (Myritol® 318 PH, Cognis GmbH, Düsseldorf, Germany) in a concentration of 1.5% (w/w) by sonication at 5×10% cycle, 100% power using ultrasound device (Bandelin, Sonopuls, HD2070) until getting a clear solution which after cooling results into a gel.

2.0 ml oleogel were added to 280.0 mg Drospirenone (micronized; Drospirenone mikro 15, Syn. C, Var. 3, ZK 30595, Stoffnummer 3123163). The mixture was been sonicated for 1 min at 1×10% cycle, 100% power using ultrasound device (Bandelin, Sonopuls, HD2070, Bandelin electronics, Berlin, Germany).

0.5 ml was filled into a 1 ml syringe (Tuberkulin 1×100 Soft-Ject, Henke Sass Wolf GmbH). A dose of 70.0 mg/0.5 ml was administered.

Sample 3.4: Polymer Particles Loaded with Drospirenone

PLGA polymer (commercially available PLGA polymer Resomer® RG 503H from Boehringer Ingelheim) and Drospirenone were mixed with a mixture of dichloromethane (DCM) and methanol (MeOH) in a 10 ml vial before adding it to a 0.4% polyvinylalcohol (PVA) 4-88 solution in a 100 ml beaker (amounts see Table 3 below).

The mixture was then emulsified for 3 hours at 500 rpm before adding it to 800 ml Millipore water in a 1000 ml beaker at 400 rpm. Subsequently, the mixture was stirred for 3 hours at 400 rpm to evaporate the dichloromethane.

The suspension with the microparticles was then filtered onto at Whatman Filter 6 using a suction strainer. The obtained filter cake was subsequently transferred and divided into two vials before each of them were resuspended in 5 ml water and freeze-dried.

TABLE 3
Content and amounts

| | Emulsion | |
|---|---|---|
| PLGA RG 503H (mg) | 100 | mg |
| Drospirenone (mg) | 150 | mg |
| Dichloromethane (ml) | 2.5 | ml |
| 0.4% PVA (ml) | 60 | ml |

### In Vivo Release Kinetics of Drospirenone

The aim of the present study was to evaluate the release of Drospirenone from subcutaneous depot formulation in rats. The present study was designed as an open, non-randomized comparison between various depot formulations containing Drospirenone used for contraception (Table 4). Pharmacokinetic parameters were calculated from the mean serum concentration-time profiles.

TABLE 4
Overview of treatments and sample origin

| | | Actual | |
|---|---|---|---|
| | Formulation | Dose | Sampling times |
| Analyte | [group] | [mg/rat] | [h] |
| Drospirenone | Aqueous MKS | 60 | 0.5, 1, 3, 6, 24, 72, |
| | (50 μm, animalgroup 1) | | 168, 336, 504, 672 |
| | Aqueous MKS | 60 | 0.5, 1, 3, 6, 24, 72, |
| | (20 μm, animalgroup 5) | | 168, 336, 504, 672 |
| | Aqueous MKS | 60 | 0.5, 1, 3, 6, 24, 72, |
| | (110 μm, animalgroup 6) | | 168, 336, 504, 672 |
| | PLGA Particles | 30 | 0.5, 1, 3, 6, 24, 72, |
| | (animalgroup 9) | | 168, 336, 504, 672 |
| | Oily MKS | 60 | 0.5, 1, 3, 6, 24, 72, |
| | (50 μm, animalgroup12) | | 168, 336, 504, 672 |

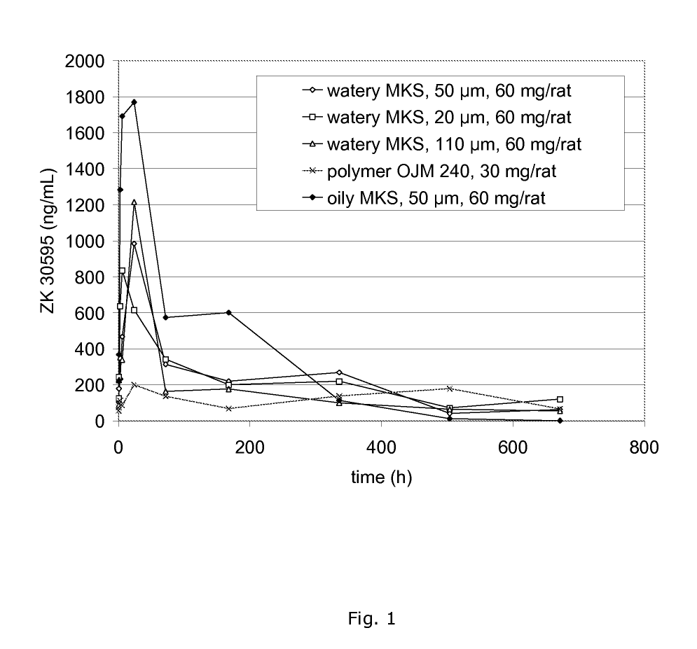

The mean pharmacokinetic parameters are summarized in FIG. 1 (Mean serum concentrations of Drospirenone after a single subcutaneous administration of Drospirenone in the above mentioned formulations).

FIG. 1 shows that a suspension of Drospirenone in peanut oil is able to provide physiological relevant levels of Drospirenone for a period of at least 15 days after subcutaneous injection of the suspension into a rat.

### Example 4

### Drospirenone/Ethinyl Estradiol Combination Formulation

### Sample 4.1: Oily Drospirenone Microcrystal Suspension Containing Ethinyl Estradiol Loaded PLGA Microparticles

Drospirenone (Drospirenone mikro 15, Syn. C, Var. 3, ZK 30595, Stoffnummer 3123163)
medium chain triglycerides (Myritol® 318 PH, Cognis GmbH, Dusseldorf, Germany)
Poly(lactide-co-gycolide) (PLGA) (commercially available PLGA polymer Resomer®RG 503H from Boehringer Ingelheim)

### Polyvinylalcohol (PVA) 4-88 (Fluka Chemie AG, Buchs, Switzerland)

1 ml medium chain triglycerides was added to 140 mg Drospirenone and about 2.5 mg ethinyl estradiol loaded PLGA microparticles. The suspension was mixed for 2 min using a vortex mixer (Heidolph, REAK 2000; level 4). Afterwards, the suspension was sonicated for 1 min at 1×10% cycle, 100% power using ultrasound device (Bandelin, Sonopuls, HD2070, Bandelin electronics, Berlin, Germany).

### Sample 4.2: Aqueous Drospirenone Microcrystal Suspension Containing Ethinyl Estradiol Loaded PLGA Microparticles

Drospirenone (Drospirenone mikro 15, Syn. C, Var. 3, ZK 30595, Stoffnummer 3123163)
Poly(lactide-co-gycolide) (PLGA) (commercially available PLGA polymer Resomer® RG 503H from Boehringer Ingelheim)

### Polyvinylalcohol (PVA) 4-88 (Fluka Chemie AG, Buchs, Switzerland)

350 mg Resomer RG 503H and 39 mg ethinyl estradiol were dissolved in 3.0 g dichlormethane. After injecting the solution into 800 ml PVA aqueous solution 0.25% it was emulsified using an Ultrathurrax at 13.000 rpm for 7 min. Thereafter, the solution was stirred for 3 h at room temperature.

The resulting microparticle dispersion was centrifuged at 2.500 rpm for 30 min and was washed 2 times with water. The sediment was redispersed in water and was thereafter lyophilized.

1 ml Millipore® water was added to 140 mg micronized drospirenone and about 2.5 mg ethinyl estradiol loaded PLGA microparticles (depending on the charge used). The suspension was mixed for 2 min using a vortex mixer (Heidolph, REAK 2000; level 4). Afterwards, the suspension was sonicated for 1 min at 1×10% cycle, 100% power using ultrasound device (Bandelin, Sonopuls, HD2070, Bandelin electronics, Berlin, Germany).

### Sample 4.3: Drospirenone Oleogel Containing Ethinyl Estradiol Loaded PLGA Microparticles

Drospirenone (Drospirenone mikro 15, Syn. C, Var. 3, ZK 30595, Stoffnummer 3123163)
Medium chain triglycerides (Myritol® 318 PH, Cognis GmbH, Dusseldorf, Germany)
Poly(lactide-co-gycolide) (PLGA) (commercially available PLGA polymer Resomer®RG 503H from Boehringer Ingelheim)

### Polyvinylalcohol (PVA) 4-88 (Fluka Chemie AG, Buchs, Switzerland)

### Excipients (E)

E.1.: methyl cholate in a concentration of 1.5% (Alfa Aesar, Karlsruhe, Germany),
E.2.: colloidal silicon dioxide in a concentration of 3% (Aerosil®200 Pharma) (Degussa, Essen, Germany)

The oleogelator was dispersed in medium chain triglycerides by sonication at 5×10% cycle, 100% power using ultrasound device (Bandelin, Sonopuls, HD2070) until getting a clear solution which after cooling results in a gel.

Thereafter, 140 mg Drospirenone and about 2.5 mg ethinyl estradiol PLGA microparticles were added to the oleogel. The mixture was been sonicated for 1 min at 1×10% cycle, 100% power using ultrasound device using (Bandelin, Sonopuls, HD2070, Bandelin electronics, Berlin, Germany).

Sample 4.4: Combination of PLGA Microparticles Loaded with Drospirenone and PBCA Microparticles Loaded Ethinyl Estradiol in the Layer
PLGA microparticles loaded with Drospirenone (prepared like described above, sample 3.4.)
Ethinyl estradiol (EE mikro 20; ZK 4944; #84303060)
N-butyl cyanoacrylate (Sicomet® 6000, Sichel Werke GmbH; Hannover, Germany)
Triton X-100 (Octoxynol 9, rein, Stoffnummer 00041327, #13051859)

### Polyvinylalcohol (PVA) 4-88 (Fluka Chemie AG, Buchs, Switzerland)

50 mg ethinyl estradiol was dissolved in 1.4 g BCA monomer mixed with a magnetic stirrer. The solution has been constantly dropped into 100 ml aqueous Triton X-100 solution 1% (w/w) acidified with hydrochloric acid (pH 2.1) at 4-7° C. under stirring with a three-blade propeller stirrer (400 rpm) over a time period of 15 min using a syringe pump. Under these conditions the dispersion has been additionally stirred for 30 min. Thereafter, the dispersion was filtered to separate particles from coarser polymer material.

Air bubbles were introduced into PBCA nanoparticle dispersion stabilized with Triton X-100 solution 1% (w/w) under moderate stirring with a three-blade propeller stirrer over a time period of 12 h by utilization of a sinter filter connected with a compressed-air supply.

For purification of drug-loaded PBCA microparticles bulk dispersion was mixed three times with 50 ml with Triton x-100 solution 1% (w/w) and separated by flotation in a separation funnel.

PBCA microparticles loaded with ethinyl estradiol and about 250 mg drospirenone loaded PLGA microparticles (depending on the charge) were dispersed in 1 ml surfactant solution (Triton X-100 0.01% and PVA 0.4%).

Sample 4.5: Combination of PLGA Microparticles Loaded with Drospirenone and PBCA Microparticles Loaded Ethinyl Estradiol in the Interior
PLGA microparticles loaded with Drospirenone (prepared like described above, sample 3.4.)
Ethinyl estradiol (EE mikro 20; ZK 4944; #84303060)
N-butyl cyanoacrylate (PBCA; Sicomet® 6000, Sichel Werke GmbH, Hannover, Germany)

### Triton X-100 (# K31255303239)

### Polyvinylalcohol (PVA) 4-88 (Fluka Chemie AG, Buchs, Switzerland)

100 g n-butyl cyanoacrylate (PBCA) (adequate to 5% (w/w)) has been added dropwise to 2000 ml 1% (w/w) Triton X-100 solution over a time period of 30 min at pH 2.1 and under cooling (4-7° C.) using a syringe pump. In the process the mixture was been moderately stirred by utilization of a three-blade propeller stirrer (300 rpm). After complete dispensing of BCA the Triton X-100 concentration was increased to 1% (w/w) and the dispersion was stirred further 30 min. Following, the sample was warmed up to room temperature and was separated from coarser polymer material by filtration (filter paper, Schleicher & Schuell GmbH, Dassel, Germany)

Air bubbles were introduced into PBCA nanoparticle dispersion stabilized with Triton X-100 solution 1% (w/w) under moderate stirring with a three-blade propeller stirrer over a time period of 12 h by utilization of a sinter filter connected with a compressed-air supply.

For purification PBCA microparticles bulk dispersion was mixed three times with 50 ml with Triton x-100 solution 0.1% (w/w) and separated by flotation in a separation funnel.

For encapsulation of ethinyl estradiol into interior of unloaded air-filled PBCA microparticles 2.5 ml of microparticle dispersion was blended in Triton X-100 solution 1%. Thereafter, 10 mg drug substance was dispersed. Following, the samples had been heated up to glass transition temperature under stirring for 15 min and were finally cooled by putting the sample into an ice bath.

For purification microparticles were mixed two times with 25 ml Triton X-100 solution 1% and once with 25 ml water and extracted by flotation in a separation funnel.

PBCA microparticles loaded with ethinyl estradiol and about 250 mg Drospirenone loaded PLGA microparticles (depending on the charge) were dispersed in 1 ml surfactant solution (Triton X-100 0.01% and PVA 0.4%).

1. A composition comprising Drospirenone dispersed in a liquid or semi-solid lipophilic vehicle.

2. A composition according to claim 1, wherein the lipophilic vehicle is semi-solid.

3. A composition according to claim 1, wherein the lipophilic vehicle is castor oil, sesame oil, peanut oil, a medium chain triglyceride or a mixture of any of these with benzyl benzoate.

4. A composition according to claim 1, wherein the concentration of Drospirenone is in the range of 20-250 mg/ml.

5. A composition according to claim 1, further comprising an Estrogen.

6. A composition according to claim 1, wherein said composition further comprises at least one excipient.

7. A composition according to claim 6 wherein the at least one excipient is methyl cholate, hydrophobic colloidal anhydrous silica, colloidal silicon dioxide, a cholesteryl fatty acid ester, polyoxyethylen(5)sorbitan monooleate, polyoxyethylen(6)stearate, a polyvalent metal salt of a fatty acid, a fatty acid ester of a carbohydrate, a sorbitan fatty acid ester, a glycerol fatty acid ester, and combinations thereof.

8. Method of preparing a composition according to claim 1, comprising the step of:
a) mixing Drospirenone and a lipophilic vehicle, or
b) dispersing Drospirenone in a mixture of lipophilic vehicle and an excipient, or
c) loading polymeric particles with Drospirenone.

9. A method for contraception comprising the step of administering by injection a composition according to claim 1 to a women.

10. A method for treating diseases, disorders or symptoms associated with deficient endogenous levels of estrogen in a woman comprising the step of administering by injection a composition according to claim 1 to a women.

11-14. (canceled)

15. A composition according to claim 1 further comprising an estrogen encapsulated in polymer particles.

16. A composition comprising Drospirenone loaded polymer particles and estrogen loaded in polymer particles in an aqueous or liquid or semi-solid lipophilic vehicle.
